SEQUENCE LISTING (1 sequence)

SEQ ID NO: 1 - protein, 323 aa, Aspergillus oryzae
ASGHGTVVTSPEPNDIEYLTPVNIGGTTLNLDFDTGSADLWVFSEELPKSEQTGHDVYKPSGNASKIAGASWDISYGDGSSASGDVYQDTVTVGGVTAQGQAVEAASKISDQFVQDKNNDGLLGLAFSSINTVKPKPQTTFFDTVKDQLDAPLFAVTLKYHAPGSYDFGFIDKSKFTGELAYADVDDSQGFWQFTADGYSVGKGDAQKAPITGIADTGTTLVMLDDEIVDAYYKQVQGAKNDASAGGYVFPCETELPEFTVVIGSYNAVIPGKHINYAPLQEGSSTCVGGIQSNSGLGLSILGDVFLKSQYVVFDSQGPRLGF

(19) United States
(12) Patent Application Publication
(10) Pub. No.: US 2023/0087917 A1
(43) Pub. Date: Mar. 23, 2023

(54) DIGESTIVE ENZYME AGENT

(71) Applicant: Amano Enzyme Inc., Nagoya-shi, Aichi (JP)

(72) Inventor: Yuki Ishigaki, Kakamigahara-shi, Gifu (JP)

(21) Appl. No.: 17/904,998

(22) PCT Filed: Feb. 26, 2021
(86) PCT No.: PCT/JP2021/007471

§ 371 Date: Aug. 25, 2022

(30) Foreign Application Priority Data
Feb. 28, 2020 (JP) 2020-034117

(51) Int. Cl. C12N 9/62; A61K 36/062; A23L 33/10; A23L 31/00; A23L 29/00

(52) U.S. Cl. CPC C12N 9/62; A61K 36/062; A23L 33/10; A23L 31/00; A23L 29/06; A61K 38/00

(57) ABSTRACT

Provided is a digestive enzyme agent which can promote the liberation of a protein into BCAAs in an in vivo environment. A digestive enzyme agent comprising a protease derived from a koji mold can promote the liberation into BCAAs in an in vivo environment.

### TECHNICAL FIELD

The present invention relates to a digestive enzyme agent capable of promoting the liberation of branched-chain amino acids, and specifically to a digestive enzyme agent comprising a protease derived from a koji mold.

### BACKGROUND ART

Leucine, isoleucine, and valine are called branched-chain amino acids (BCAAs), and exhibit useful effects such as suppression of muscle proteolysis and promotion of protein synthesis. In order to efficiently enjoy these useful effects, active ingestion or administration of BCAAs is regarded as important.

From such a viewpoint, a method for preparing a proteolytic product highly containing a BCAA has been studied. For example, Patent Document 1 discloses a method for producing a fraction containing a branched amino acid at a high content, in which a proteolytic, product containing a branched-chain amino acid is placed in an aqueous environment having a polar organic solvent concentration of 70 v/v % or more, and then a precipitate is removed to recover a soluble fraction, and discloses that, as a specific method for producing a proteolytic product containing a branched-chain amino acid, a glycine decomposition product is obtained by subjecting a soybean glycine composition solution to a reaction with Thermoase, a reaction with Bioprase, and a reaction with Sumizyme FP, each at 58° C. for 60 minutes.

Patent Document 2 discloses a method for producing a protein synthesis promoter including a hydrolysis step that hydrolyzes a whey protein while thermally denaturing the whey protein at a pH of 6 to 10 and a temperature of 50 to 70° C. using a heat-resistant protein hydrolase, and an inactivation step that inactivates the enzyme by heating after the hydrolysis step, and specifically, discloses that the pH of a whey protein aqueous solution is adjusted to a pH of 8 by adding papain, the whey protein is enzymatically decomposed while being denatured at 55° C. for 6 hours, the enzyme is then inactivated, and a dried product of a centrifugation supernatant thus obtained is used to obtain a whey protein hydrolysate having a high BCAA content.

### PRIOR ART DOCUMENT

### Patent Documents

Patent Document 1: WO 2008/123033 A

Patent Document 2: WO 2011/108692 A

### SUMMARY OF THE INVENTION

### Problems to Be Solved By the Invention

However, since the above-described method for preparing a proteolytic product is based on the premise that the prepared decomposition product itself or a specific fraction obtained from the decomposition product is ingested, it is necessary to perform the decomposition itself of the protein by heating the protein to a temperature exceeding a body temperature in advance outside the body (for example, a factory) using papain, Thermoase, or the like. Therefore, the enzyme used in these methods cannot support the digestion of the ingested food into BCAAs in the body.

Therefore, an object of the present invention is to provide a digestive enzyme agent capable of promoting the liberation of a protein into BCAAs in an in vivo environment.

### Means for Solving the Problem

The present inventor has conducted intensive studies, and as a result, has found that a protease derived from a koji mold is an effective component as a digestive enzyme agent capable of promoting the liberation of a protein into BCAAs in an in vivo environment. The present invention has been completed based on these findings.

That is, the present invention provides inventions of the following aspects.

Item 1. A digestive enzyme agent comprising a protease derived from a koji mold.

Item 2. The digestive enzyme agent described in Item 1, in which the koji mold is Aspergillus oryzae and/or Aspergillus niger.

Item 3. The digestive enzyme agent described in Item 1 or 2, in which the protease comprises an acidic protease.

Item 4. The digestive enzyme agent described in Item 3, in which the acidic protease is used in an amount of 10 U or more per 1 g of a substrate protein.

Item 5. The digestive enzyme agent described in any one of Items 1 to 4, in which the digestive enzyme agent is used for digestion of meat.

Item 6. The digestive enzyme agent described in any one of Items 1 to 4, in which the digestive enzyme agent is used for digestion of a vegetable protein.

Item 7. A drug for oral administration for promoting liberation of a protein into branched-chain amino acids, comprising the digestive enzyme agent described in any one of Items 1 to 6.

Item 8. A food additive for promoting liberation of a protein into branched-chain amino acids, comprising the digestive enzyme agent described in any one of Items 1 to 6.

Item 9. A food or drink for promoting liberation of a protein into branched-chain amino acids, comprising the digestive enzyme agent described in any one of Items 1 to 6.

### Advantages of the Invention

According to the present invention, there is provided a digestive enzyme agent capable of promoting the liberation of a protein into BCAAs in an in vivo environment.

### EMBODIMENTS OF THE INVENTION

A digestive enzyme agent of the present invention is characterized by comprising a specific protease. Hereinafter, the digestive enzyme agent of the present invention will be described in detail.

### Protease Derived from Koji Mold

The digestive enzyme agent of the present invention comprises a protease derived from a koji mold as an active ingredient. The koji mold from which the protease is derived is not particularly limited, and examples thereof include bacteria of the genus Aspergillus and the bacteria of the genus Rhizopus. Specific examples of the bacteria of the genus Aspergillus include Aspergillus oryzae, Aspergillus niger, Aspergillus awamori, Aspergillus kawachii, Aspergillus saitoi, Aspergillus inuii, Aspergillus sojae, Aspergillus tamari, Aspergillus glaucus, Aspergillus melleus, Aspergillus aculeates, Aspergillus caesiellus, Aspergillus candidus, Aspergillus carneus, Aspergillus clavatus, Aspergillus deflectus, Aspergillus fischerianus, Aspergillus fumigants, Aspergillus nidulans, Aspergillus parasiticus, Aspergillus penicilloides, Aspergillus restrictus, Aspergillus sydowii, Aspergillus terreus, Aspergillus ustus, and Aspergillus versicolor. Specific examples of the bacteria of the genus Rhizopus include Rizopus oryzae.

In the digestive enzyme agent of the present invention, as for the koji mold-derived protease, those derived from one of these koji molds may be used alone, or those derived from a plurality of species may be used in combination.

In the digestive enzyme agent of the present invention, among the koji molds from which the protease is derived, from the viewpoint of obtaining a further higher effect of promoting the liberation of BCAAs from a protein, Aspergillus oryzae, Aspergillus niger, Aspergillus melleus, and/or Rizopus oryzae are preferably mentioned, Aspergillus oryzae, Aspergillus niger, and/or Rizopus oryzae are more preferably mentioned, and Aspergillus oryzae is further preferably mentioned.

The type of protease is not particularly limited as long as it is an exo-type protease, and examples thereof include an acidic protease and a neutral protease. Among these proteases, from the viewpoint of obtaining a further higher effect of promoting the liberation of BCAAs from a protein, an acidic protease is preferably mentioned. That is, the koji mold-derived protease contained in the digestive enzyme agent of the present invention preferably comprises at least an acidic protease.

In the digestive enzyme agent of the present invention, the combination of the type of the koji mold from which the koji mold-derived protease is derived and the type of the protease is arbitrary. Among these arbitrary combinations, from the viewpoint of further efficiently obtaining an effect of promoting the liberation of BCAAs from a protein, an Aspergillus oryzae-derived acidic protease, an Aspergillus niger-derived acidic protease, and/or a Rizopus oryzae-derived acidic protease are preferably mentioned, and an Aspergillus oryzae-derived acidic protease is more preferably mentioned.

Specific examples of the Aspergillus oryzae-derived acidic protease include polypeptides shown in any of the following (1) to (3).

(1) A polypeptide consisting of an amino acid sequence shown in SEQ ID NO: 1.

(2) A polypeptide comprising an amino acid sequence in which one or a few amino acids are substituted, added, inserted, or deleted in the amino acid sequence shown in SEQ ID NO: 1, and having a BCAA liberation ability equivalent to that of a polypeptide consisting of an amino acid sequence shown in SEQ ID NO: 1.

(3) A polypeptide comprising an amino acid sequence having 80% or more sequence identity to an amino acid sequence shown in SEQ ID NO: 1, and having a BCAA liberation ability equivalent to that of a polypeptide consisting of an amino acid sequence showy in SEQ ID NO: 1.

The polypeptide set forth in the above (1) is a wild-type Aspergillus oryzae-derived acidic protease, and the polypeptides set forth in the above (2) and (3) are mutant-type Aspergillus oryzae-derived acidic proteases. All of these polypeptides are excellent in substrate specificity for recognizing an amino acid residue portion corresponding to a BCAA of the protein, and thus exhibit an excellent effect of promoting the liberation of BCSAA.

In the polypeptide of the above (2), amino acid modifications introduced may comprise any one of the modifications including substitution, addition, insertion, and deletion alone (for example, substitution alone) or comprise two or more of the modifications (for example, substitution and insertion). In the polypeptide of the above (2), the number of amino acids which is substituted, added, inserted, or deleted may be one or a few, and is, for example, 1 to 81, preferably 1 to 48 or 1 to 32, further preferably 1 to 16, 1 to 10, or 1 to 8, and particularly preferably 1 to 3, 1 or 2, or 1.

In the polypeptide of the above (3), sequence identity to the amino acid sequence shown in SEQ ID NO: 1 may be 80% or more, and is preferably 85% or more, preferably 90% or more, further preferably 95% or more, and particularly preferably 99% or more.

Herein, in the polypeptide of the above (3), the sequence identity to the amino acid sequence shown in SEQ ID NO: 1 refers to a sequence identity calculated by comparison with the amino acid sequence shown in SEQ ID NO: 1. The “sequence identity” refers to a value of amino acid sequence identity obtained by bl2seq program (Tatiana A. Tatsusova, Thomas L. Madden, FEMS Microbiol. Lett., Vol. 174, p 247-250, 1999) in BLAST PACKAGE [sgi32 bit edition, Version 2.0.12; available from National Center for Biotechnology Information (NCBI)]. Parameter settings may be as follows: Gap insertion Cost value: 11 and Gap extension Cost value: 1.

In the polypeptides of the above (2) and (3), when an amino acid substitution is introduced in the amino acid sequence shown in SEQ ID NO: 1, examples of a preferred aspect of the amino acid substitution introduced include a conservative substitution. That is, examples of the substitution in the polypeptides of the above (2) and (3) include the following substitutions: when an amino acid to be substituted is a non-polar amino acid, a substitution with other non-polar amino acids; when an amino acid to be substituted is a non-charged amino acid, a substitution with other non-charged amino acids; when an amino acid to be substituted is an acidic amino acid, a substitution with other acidic amino acids; and when an amino acid to be substituted is a basic amino acid, a substitution with other basic amino acids.

In the polypeptides of the above (2) and (3), the phrase “having a BCAA liberation ability equivalent to that of a polypeptide consisting of an amino acid sequence shown in SEQ ID NO: 1” refers to showing a BCAA liberation ability evaluation value equivalent to that of the polypeptide of the above (1) when the BCAA liberation ability evaluation values are measured under the following method (that is, showing a BCAA liberation ability evaluation value of the polypeptide of (2) or (3) of about 30 to 170%, 50 to 150%, or 80 to 120% when the BCAA liberation ability evaluation value of the polypeptide of the above (1) is regarded as 100%).

### BCAA Liberation Ability Measurement Method

Per 13 g of finely ground (3 mm ground) beef thigh meat (protein content: about 20 wt %), 100 mL of artificial gastric juice (50 mmol/L NaCl, 2 mmol/L KCl, 0.18 mmol/L CaCl₂, 13% McIlivaine buffer solution (pH 5.0)) is allowed to stand in a boiling water bath for 10 minutes. Thereafter, the mixture is allowed to stand at 37° C. for 30 minutes, and the polypeptide of (1), (2), or (3) corresponding to 5,000 U of protease activity (pH 3.0) per 13 g of the protein is added, and the mixture is stirred at 250 rpm for 90 minutes. From 5 minutes after the start of the reaction to 65 minutes of the reaction, 0.54 mL of 1 mol/L hydrochloric acid is added every 10 minutes. After 90 minutes, the mixture is allowed to stand in the boiling water bath for 10 minutes. After standing in the boiling water bath, the reaction solution is subjected to mesh filtration (2 mm square), and the obtained filtrate is centrifuged at 10,000 rpm for 10 minutes, thereby obtaining a supernatant containing free amino acids. As a control, the same operation is performed except that the polypeptide of the above (1), (2), or (3) is not added, thereby obtaining a supernatant. The obtained supernatant is diluted 25-fold with water and filtered through a filter (0.45 μm), and then the amount of free amino acids is measured with an amino acid analyzer. The proportion (%) of the amount (mg/L) of free BCAAs in the total amount (mg/L) of free amino acids is obtained as the BCAA liberation ability evaluation value. The BCAA liberation ability evaluation value reflects the degree of substrate specificity for recognizing an amino acid residue portion corresponding to a BCAA of the protein.

The content of the acidic protease in the digestive enzyme agent of the present invention is not particularly limited, and is, for example, 1,000 U/g or more. From the viewpoint of further efficiently obtaining the effect of promoting the liberation of BCAAs from a protein, the content of the acidic protease in the digestive enzyme agent of the present invention is preferably 3,000 to 400,000 U/g in terms of an acidic protease activity value at a pH of 3 measured by the following method.

When the digestive enzyme agent of the present invention comprises an acidic protease and a neutral protease, the acidic protease can he contained so that the ratio of the acidic protease activity at a pH of 3 measured by the following method to the neutral protease activity at a pH of 6 measured by the following method is, for example, 0.027 or more.

From the viewpoint of further efficiently obtaining the effect of promoting the liberation of BCAAs from a protein, in the digestive enzyme agent of the present invention comprising an acidic protease and a neutral protease, the ratio of the acidic protease activity at a pH of 3 of the acidic protease to the neutral protease activity at a pH of 6 is preferably as large as possible, and is preferably 0.09 or more or 0.5 or more, more preferably 0.7 or more, further preferably 1 or more, even more preferably 1.3 or more, 1.5 or more, or 2.0 or more, and particularly preferably 2.2 or more.

### Method for Measuring Protease Activity (Casein-Folin Method)

First, in a test tube, 5 mL of a 6.0 g/L casein solution (pH 3.0 when acidic protease activity is measured, and pH 6.0 when neutral protease activity is measured) is placed and maintained at 37° C. Then, 1 mL of a digestive enzyme agent aqueous solution obtained by n-fold dilution of a digestive enzyme agent to be measured for protease activity is added and allowed to stand at 37° C. for exactly 10 minutes, and then 5 mL of a 0.44 mol/L trichloroacetic acid solution is added to stop the reaction. The mixture is allowed to stand at 37° C. for 30 minutes followed by filtration with filter paper to obtain 2 mL of filtrate, which is transferred into another test tube, and then 5 mL of 0.55 mon sodium carbonate and 1 mL of 3-fold diluted Folin's reagent are added thereto in this order. The mixture is allowed to stand at 37° C. for 30 minutes followed by measurement of absorbance at an absorption wavelength of 660 nm. As a blank operation, a trichloroacetic acid solution is added to a digestive enzyme agent aqueous solution, a casein solution is then added thereto, and the mixture is allowed to stand at 37° C. for 30 minutes followed by measurement of absorbance at an absorption wavelength of 660 nm. Separately, a standard curve for tyrosine is constructed using 10 to 40 μg/mL of tyrosine solutions by the same operation as the above-described operation for the filtrate. Under the above conditions, an amount of an enzyme which causes an increase in colored materials by Folin's reagent corresponding to 1 μg of tyrosine per minute at 37° C. is defined as 1 U. The following equation is used for the calculation.

Protease activity per 1 g of sample (U/g)=(At−Ab)×F×5.5×0.1×n [Mathematical Formula 1]

At: Absorbance of enzyme reaction solution

Ab: Absorbance of blank solution

F: Amount (μg) of tyrosine corresponding to difference in absorbance of 1 as determined by standard curve for tyrosine

5.5: Factor of conversion after completion of reaction into total liquid amount

0.1: Factor of conversion into per minute of reaction

n: Dilution factor

The digestive enzyme agent comprising the protease derived from a koji mold described above may be produced using a koji mold producing the protease, or may be produced by a known genetic engineering technique, and commercially available products may be used. In the case of using a commercially available product, examples of the digestive enzyme agent comprising an Aspergillus oryzae-derived acidic protease so that the acidic protease activity at a pH of 3 with respect to the neutral protease activity at a pH of 6 is relatively large include ASPSDU-pine, Protease M Amano SD, Peptidase R, Acidic protease UF Amano SD (all manufactured by Amano Enzyme Inc.), Orientase AY (manufactured by HBI Enzymes Inc.), and PROTEASE YP-SS (manufactured by Yakult Pharmaceutical Industry Co., Ltd.); and examples of the digestive enzyme agent comprising an Aspergillus oryzae-derived neutral protease so that the acidic protease activity at a pH of 6 with respect to the neutral protease activity at a pH of 3 is relatively large include ProteAX, Protease P Amano 3SD (all manufactured by Amano Enzyme Inc.), and Sumizyme (manufactured by SHINNIHON CHEMICALS Corporation).

The content of the protease derived from a koji mold in the enzyme preparation of the present invention is appropriately set in a range in which the effect of promoting the liberation of BCAAs by the protease derived from a koji mold is exhibited.

### Other Ingredients

The digestive enzyme agent of the present invention may comprise, in addition to the above-described active ingredient, microbial cell component of a koji mold producing the above-described active ingredient, other nutrient components, pharmacological components, and/or enzyme components as necessary. The nutrient components, the pharmacological components, and the enzyme components are not particularly limited as long as they can be used in the food or drink and/or the drug, and examples thereof include vitamins such as vitamin B1, vitamin B2, vitamin B6, vitamin B12, vitamin C, vitamin A, vitamin D, vitamin E, vitamin K, niacin, pantothenic acid, folic acid, biotin, and lycopene; minerals such as calcium, sulfur, magnesium, zinc, selenium, and iron; proteolytic products; amino acids such as BCAAs (leucine, isoleucine, and valine), glycine, alanine, arginine, aspartic acid, cystine, phenylalanine, taurine, and tryptophan; fatty acids such as linoleic acid, γ-linolenic acid, α-linolenic acid, docosahexaenoic acid, and eicosapentaenoic acid; herbal medicines; plant extracts; other functional materials such as dietary fiber, rotary jelly, propolis, honey, chondroitin sulfate, glucosamine, ceramide, and hyaluronic acid; stomachics such as betaine hydrochloride, carnitine chloride, and betanequol chloride; antiflatulents; carbohydrate digestive enzymes such as amylase, glucosidase, galactosidase, glucoamylase, maltase, and cellulase; lipid-degrading enzymes such as lipase; protein digestive enzymes such as peptidase, nattokinase, and protease other than the above-described active ingredients (koji mold-derived protease); and other enzymes such as phosphatase, nuclease, deaminase, oxidase, dehydrogenase, glutaminase, pectinase, catalase, dextranase, transglutaminase, protein deamidase, and pullulanase.

These nutrient components, pharmacological components, and/or enzyme components may be used singly or in combination of two or more kinds thereof. The content of these components is appropriately set depending on the type of the component to be used, the form and/or use application of the digestive enzyme agent of the present invention, and the like.

The digestive enzyme agent of the present invention may comprise a base and/or an additive or the like as necessary in order to prepare the digestive enzyme agent into a desired preparation form. Such a base and an additive are not particularly limited as long as they can be used for foods or drinks and/or drugs, and examples thereof include diluents (such as starch, dextrin, maltose, trehalose, lactose, D-glucose, sorbitol, D-mannitol, sucrose, and glycerol), buffers (such as phosphate, citrate, and acetate), stabilizers (such as propylene glycol and ascorbic acid), preservatives (sodium chloride, phenol, benzalkonium chloride, benzyl alcohol, chlorobutanol, and methylparaben), antiseptics (such as sodium chloride, ethanol, benzalkonium chloride, paraoxybenzoic acid, and chlorobutanol), water, alcohols, fats and oils, water-soluble polymers, surfactants, pH adjusters, ultraviolet inhibitors, flavoring agents, thickeners, dyes, and chelating agents.

These bases and/or additives may be used singly or in combination of two or more kinds thereof. The content of these bases and/or additives is appropriately set depending on the type of the agent to be used, the form and/or use application of the digestive enzyme agent of the present invention, and the like.

### Usage and Dose

The digestive enzyme agent of the present invention is orally ingested or orally administered. The timing of ingestion or administration of the digestive enzyme agent of the present invention is not particularly limited as long as the ingested substrate protein and the ingested or administered digestive enzyme agent of the present invention coexist in the body, and is, for example, during a meal, before a meal, or after a meal.

The dose of the digestive enzyme agent of the present invention can be appropriately set depending on the type of protease products in which the agent is used, use applications, the amount of substrate protein, expected effects, dosage forms, and the like.

The ingestion or dosage amount of the digestive enzyme agent of the present invention per meal containing proteins varies depending on the ingestion amount of the substrate protein, and is, for example, 1 to 2,000 mg, 2 to 1,000 mg, 3 to 500 mg, or 5 to 400 mg.

Specifically, the ingestion or dosage amount of the digestive enzyme agent of the present invention per meal containing proteins varies depending on the ingestion amount of the substrate protein, and is, as the amount of the acidic protease, for example, 100 U or more. From the viewpoint of obtaining a further higher effect of promoting the liberation of BCAAs from a protein, the ingestion or dosage amount of the digestive enzyme agent of the present invention per meal containing proteins is, as the amount of the acidic protease, preferably an amount of 200 U or more, 500 U or more, 1,000 U or more, 2,000 U or more, 5,000 U or more, 10,000 U or more, 20,000 U or more, or 30,000 U or more, The upper limit of the range of the amount of the acidic protease is not particularly limited, and is, for example, 400,000 U or less, 200,000 U or less, 100,000 U or less, or 80,000 U or less.

More specifically, in the digestive enzyme agent of the present invention, the acidic protease can he used in an amount of, for example, 10 U or more per 1 g of the substrate protein. From the viewpoint of obtaining a further higher effect of promoting the liberation of BCAAs from a protein, as for the amount of the acidic protease per 1 g of the substrate protein in the digestive enzyme agent of the present invention, for example, it is preferable to use the acidic protease per 1 g of the substrate protein in an amount of 20 U or more, preferably 50 U or more, more preferably 100 U or more, further preferably 200 U or more, even more preferably 500 U or more, and particularly preferably 800 U or more, 1,000 U or more, 1,500 U or more, or 1,800 U or more. From the viewpoint of obtaining a further higher effect of promoting the liberation of BCAAs from a protein, in the digestive enzyme agent of the present invention, the amount of the acidic protease to be used per 1 g of the substrate protein may be 1,800 or more, 2,000 U or more, 2,500 U or more, 3,000 U or more, or 5,000 U or more.

As for the amount of the digestive enzyme agent of the present invention used, the upper limit of the amount of the acidic protease per 1 g of the substrate protein is not particularly limited, and is, for example, 20,000 U or less, 10,000 U or less, or 7,000 U or less. From the viewpoint of efficiently obtaining an effect of promoting the liberation of BCAAs with respect to the amount of the enzyme agent used, the upper limit of the amount of the acidic protease per 1 g of the substrate protein may be, for example, 6,000 U or less, 5,000 U or less, 3,000 U or less, 2,000 U or less, 1,500 U or less, or 1,000 U or less.

### Use Application

The digestive enzyme agent of the present invention is used for the purpose of promoting the liberation of branched-chain amino acids (BCAAs) from a substrate protein by the action of the protease as an active ingredient. In the present invention, promoting the liberation of BCAAs refers to liberating a larger amount of BCAAs than the BCAA liberation amount by a protease other than a protease derived from a koji mold, by digestion, and in a preferred embodiment, liberating BCAAs so that the total amount of free BCAAs to the total amount of free amino acids is larger than the ratio of the BCAA residues to the total amount of amino acid residues in the substrate. That is, the digestive enzyme agent of the present invention can be used as a liberation promotor of a protein into BCAAs.

The digestive enzyme agent of the present invention can promote the liberation of BCAAs in an in vivo environment. Therefore, the digestive enzyme agent of the present invention can be used for the purpose of performing digestion in an environment of, for example, 35 to 40° C., preferably 35.5 to 38° C., more preferably 36 to 37.5° C., and further preferably 36.5 to 37.5° C. Particularly preferably, the digestive enzyme agent of the present invention can he used for purpose of supporting digestion in the digestive organs.

The pH to he applied at the time of digestion of the digestive enzyme agent of the present invention varies depending on the type and content ratio of the protease to he contained, but in the case of comprising an acidic protease as a preferred embodiment at a predetermined ratio, the digestive enzyme agent of the present invention can be used fir the purpose of digestion in an environment of a pH of 1 to 6.5, preferably a pH of 1.5 to 5, more preferably a pH of 2 to 4.5, further preferably a pH of 2.5 to 4, and even more preferably a pH of 2.5 to 3.5. Therefore, the digestive enzyme agent of the present invention can be preferably used for purpose of supporting digestion in the stomach.

The digestive enzyme agent of the present invention can be used for the purpose of digesting any protein. Therefore, the digestive enzyme agent of the present invention can be used for the purpose of digesting animal proteins such as meat, fish and shellfish, and dairy products; and vegetable proteins such as wheat, beans, and nuts.

The digestive enzyme agent of the present invention can be used for the purpose of digesting a protein having a high content of BCAAs in order to promote the liberation of BCAAs. Since the digestive enzyme agent of the present invention has excellent digestibility capable of liberating not only BCAAs but also a large amount of total amino acids, the digestive enzyme agent of the present invention can be used for the purpose of digesting protein foods which are difficult to digest by itself. From these viewpoints, preferred examples of protein foods to which the digestive enzyme agent of the present invention is applied include meat (meat of livestock). Specific examples of the meat include meat of animals of mammals such as cows, pigs, horses, sheep, boars, deer, and whales; and birds such as chickens, ducks, piglets, and quails, preferably include meat of mammals, and more preferably include meat of cows. The site of the animal is not particularly limited, and examples thereof include neck, back, abdomen, thigh, shank, buttocks, and preferably include thigh.

Since the digestive enzyme agent of the present invention is excellent in the effect of promoting the liberation of BCAAs, the digestive enzyme agent of the present invention can liberate a large amount of BCAAs even from a vegetable protein food having a relatively low protein content. Preferable examples of the vegetable protein food include wheat, beans, and nuts, more preferable examples thereof include beans, further preferable examples thereof include peas and soybeans, even more preferable examples thereof include soybeans, and particularly preferable examples thereof include green soybeans.

According to the digestive enzyme agent of the present invention, since the liberation of BCAAs can be promoted, the digestive enzyme agent of the present invention can be used for a subject requiring active ingestion of BCAAs. Examples of such a subject include subjects requiring suppression of muscle proteolysis and/or promotion of muscle protein synthesis, and specifically include subjects requiring inhibition of muscle fatigue, improvement of muscle damage, muscle enhancement, and the like. Since the digestive enzyme agent of the present invention has excellent digestibility capable of liberating not only BCAAs but also a large amount of total amino acids, the digestive enzyme agent of the present invention can be used not only for subjects requiring active ingestion of BCAAs but also for subjects requiring support of digestion. Examples of such a subject include subjects during or after illness and elderly subjects (in the case of humans, for example, 60-years-old or older).

Examples of the subject to which the digestive enzyme agent of the present invention is applied include humans and non-human mammals, Examples of the non-human mammals include experimental animals such as mice, rats, rabbits, guinea pigs, and primates other than humans; pet animals (pets) such as dogs and cats; livestock such as cattle, pigs, goats, sheep, and horses; and humans. Among these application targets, humans, pet animals, and livestock are preferably mentioned, and humans are more preferably mentioned.

### Dosage Form, Formulation Form, and Usage

The digestive enzyme agent of the present invention is used for promoting the liberation of BCAAs during the digestion of proteins under in vivo conditions or under in vitro conditions that simulates an in vivo environment. Therefore, the digestive enzyme agent of the present invention is formulated as an oral enzyme agent or enzyme reagent. Particularly preferably, the digestive enzyme agent of the present invention is formulated as an oral enzyme agent, specifically, an oral enzyme agent by oral ingestion or oral administration.

The formulation form of the digestive enzyme agent of the present invention is not particularly limited, and can be appropriately determined by those skilled in the art depending on the use form. A specific embodiment when the digestive enzyme agent of the present invention is formulated as an oral enzyme agent is not particularly limited as long as it can be orally ingested or orally administered, and specific examples thereof include a food or drink, a food additive, and a drug for oral administration.

When the digestive enzyme agent of the present invention is prepared in the form of a food or drink, the above-described active ingredient may be prepared as it is or in a desired form in combination with the above-described other ingredients, other food materials, and/or seasoning. Examples of such a food or drink include, in addition to general foods or drinks, foods for specified health use, foods with function claims, dietary supplements, foods for patients, and foods for the elderly. Examples of such a food or drink include not only a food or drink for human but also a feed for experimental animals or livestock, and a pet food for pen animals. The form of these foods or drinks is not particularly limited, and specific examples thereof include supplements such as capsules (soft capsules and hard capsules), tablets, granules, powders, and jellies; beverages such as nutritious drinks, fruit juice beverages, carbonated beverages, and lactic acid beverages; and items of personal preference such as dumpling, ice, sherbet, gummy, and candy. Among these foods or drinks, supplements are preferably mentioned, and capsules, tablets, granules, and powders are more preferably mentioned. These foods or drinks are suitably used as foods or drinks for promoting the liberation of a protein into branched-chain amino acids.

When the digestive enzyme agent of the present invention is prepared in the form of a food additive, the above-described active ingredient may be prepared as it is or in a desired form in combination with the above-described other ingredients and/or seasoning. Examples of such a food additive include not only those added to a food or drink for human but also those added to a feed for experimental animals or livestock, and those added to a pet food for pm animals. Examples of the form of such a food additive include granules, powders, and solutions which are easily mixed with food, and from the viewpoint of stability, granules and powders are preferably mentioned. These food additives are suitably used as food additives for promoting the liberation of a protein into branched-chain amino acids.

When the digestive enzyme agent of the present invention is prepared in the form of a drug for oral administration, the above-described active ingredient may be prepared as it is or in a desired form in combination with the above-described other ingredients. Specific examples of such a drug for oral administration include capsules (soft capsules and hard capsules), tablets, granules, powders, jellies, and syrups. Among these drugs tor oral administration, capsules, tablets, granules, and powders are preferably mentioned. These drugs for oral administration are suitably used as drugs for oral administration for promoting the liberation of a protein into branched-chain amino acids.

When the digestive enzyme agent of the present invention is configured as a drug for oral administration, the drug for oral administration can be taken before, simultaneously with or after a meal of a food containing a protein, can be preferably taken after a meal, and can be more preferably taken within 20 to 40 minutes alter a meal.

When the digestive enzyme agent of the present invention is prepared in the form of an enzyme reagent, the above-described active ingredient may be prepared as it is or in a desired form in combination with the above-described other ingredients. Examples of the form of such an enzyme reagent include granules, powders, and solutions which are generally easy to construct a protein digestive system in vitro, and from the viewpoint of stability, granules and powders are preferably mentioned. These enzyme reagents are suitably used as enzyme reagents for promoting the liberation of a protein into branched-chain amino acids.

When the digestive enzyme agent of the present invention is configured as an enzyme reagent, the enzyme reagent can be used for testing the promotion of liberation of BCAAs from a protein in an in vivo environment, preferably in an artificial digestive system constructed by simulating the intragastric environment, specifically in an artificial digestive system containing artificial gastric juice and adjusted to a temperature condition corresponding to a body temperature, and the enzyme reagent can be added to the artificial digestive system, for example, before, simultaneously with or after feeding the protein to the artificial digestive system, more preferably within 20 to 40 minutes after feeding the protein to the artificial digestive system.

The content of the digestive enzyme agent in these oral enzyme agents or enzyme reagents is appropriately set in an amount range in which the protease derived from a koji mold contained in the digestive enzyme agent exhibits an effect of promoting the liberation of BCAAs by the protease derived from a koji mold.

### EXAMPLES

Hereinafter, the present invention will be specifically described by means of Examples; however, the present invention is not to he construed as being limited to the following Examples.

### Test Example

### Test Substrate

As a test substrate, beef thigh meat (lean tissue) was prepared. The weight ratio of BCAAs to the total amino acid weight of the beef thigh meat (lean tissue) is 22.9%. The amount of the beef thigh meat used was set to 13 g for each protease, and the beef thigh meat was finely ground (3 mm ground).

### Digestive Enzyme Agent

Digestive enzyme agents shown in Table 1 were prepared. The amount of the digestive enzyme agent used was set so that the protease activity would be an amount of 3000 U as measured by an enzyme activity measurement method (measurement pH: 6.0) based on the following Folin method. For the digestive enzyme agent of 3000 U of the protease, the activity as measured by an enzyme activity measurement method (measurement pH: 3.0) based on the following Folin method was obtained as the acidic protease activity (unit: U). The ratio of the acidic protease activity (unit: U) to 3000 U of the protease measured at a pH of 6.0 was regarded as the acidic protease ratio. These measurement results are shown in Table 1. In Table 1, the acidic proteases contained in the digestive enzyme agents shown in Examples 1 to 3 is a polypeptide consisting of an amino acid sequence shown in SEQ ID NO: 1; a polypeptide comprising an amino acid sequence in which one or a few amino acids are substituted, added, inserted, or deleted in the amino acid sequence shown in SEQ ID NO: 1, and having a protease activity equivalent to that of a polypeptide consisting of an amino acid sequence shown in SEQ ID NO: 1; or a polypeptide comprising an amino acid sequence having 80% or more sequence identity to an amino acid sequence shown in SEQ ID NO: 1, and having a protease activity equivalent to that of a polypeptide consisting of an amino acid sequence shown in SEQ ID NO: 1.

### Enzyme Activity Measurement Method (Casein-Folin Method)

In a test tube, 5 mL of a measurement substrate solution adjusted to a predetermined measurement pH (when the measurement pH was 3.0, an aqueous solution at a pH of 3.0 containing 6.0 g of milk casein and 0.08 mol/L of lactic acid; when the measurement pH was 6.0, an aqueous solution at a pH of 6.0 containing 6.0 g/L of milk casein and 0.04 mol/L of disodium phosphate) was placed and maintained at 37° C. for 10 minutes, Subsequently, 1 mL of a digestive enzyme agent aqueous solution diluted to an appropriate concentration (n-fold dilution) was added to the test tube and immediately shaken up, the mixture was allowed to stand at 37° C. for exactly 10 minutes, and then 5 mL of a 0.44 mol/L trichloroacetic acid solution was added and shaken up to stop the reaction. The mixture was allowed to stand at 37° C. for 30 minutes followed by filtration with filter paper to obtain 3 mL of the first filtrate, which was removed, and 2 mL of the next filtrate, which was transferred into another test tube, then 5 mL of a 0.55 mol/L sodium carbonate solution and 1 mL of an aqueous solution obtained by 3-fold dilution of a commercially available Folin's reagent were added thereto in this order, and the mixture was shaken up thoroughly and allowed to stand at 37° C. for 30 minutes. The absorbance At of this solution at a wavelength of 660 nm was measured using water as a control.

Separately, as a blank operation, 5 mL of a 0.44 mol/L trichloroacetic acid solution was added to 1 mL of a digestive enzyme agent aqueous solution and shaken up, 5 mL of a measurement substrate solution was then added and immediately shaken up, and the mixture was allowed to stand at 37° C. for 30 minutes. The absorbance At of this solution at a wavelength of 660 nm was measured using water as a control.

A standard curve for tyrosine was constructed using 10 to 40 μg/mL of tyrosine solutions by the same operation as the above-described operation for the filtrate. Under the conditions of the enzyme activity measurement method described above, an amount of an enzyme which causes an increase in colored materials by Folin's reagent corresponding to 1 μg of tyrosine per minute at 37° C. was defined as 1 U. The following equations was used for the calculation.

Protease activity per 1 g of sample (U/g)=(At−Ab)×F×5.5×0.1×n [Mathematical Formula 2]

At: Absorbance of enzyme reaction solution

Ab: Absorbance of blank solution

F: Amount (μg) of tyrosine corresponding to difference in absorbance of 1 as determined by standard curve for tyrosine

5.5: Factor of conversion after completion of reaction into total liquid amount

0.1: Factor of conversion into per minute of reaction

n: Dilution factor

### Digestion Experiment in Digestive System Simulating Stomach

In a 100 mL Erlenmeyer flask, 13 g of beef thigh meat as a test substrate and 100 mL of artificial gastric juice (50 mmol/L NaCl, 2 mmol/L KCl, 0.18 mmol/L CaCl₂, 13% McIlvaine buffer solution (pH 5.0)) were placed and allowed to stand in a boiling water bath for 10 minutes. Thereafter, the mixture was allowed to stand at 37° C. for 30 minutes, a digestive enzyme agent corresponding to 3,000 U of protease activity (pH 6.0) was added thereto, a stirrer bar (3.5 cm) was put thereinto, and the mixture was stirred at 250 rpm for 90 minutes. From 5 minutes to 65 minutes after the start of the reaction, 0.54 mL of 1 mol/L hydrochloric acid was added every 10 minutes. After 90 minutes, the mixture was allowed to stand in the boiling water bath for 10 minutes. After standing in the boiling water bath, the reaction solution was subjected to mesh filtration (2 mm square), and the obtained filtrate was centrifuged at 10,000 rpm for 10 minutes, thereby obtaining a supernatant containing free amino acids. As a control (Comparative Example 1), the same operation was performed except that the digestive enzyme agent was not added, thereby obtaining a supernatant.

The obtained supernatant was diluted 25-fold with water and filtered through a filter (0.45 μm), and then the amount of free amino acids was analyzed with an amino acid analyzer (amino acid analysis using Agilent 1260 infinity II LC system) according to the protocol. The total amount (mg/L) of free amino acids thus obtained and the proportion (%) of the amount (mg/L) of free BCAAs in the total amount (mg/L) of free amino acids are shown in Table 1.

TABLE 1
Substrate: beef thigh meat 13 g (substrate protein: 2.73 g), reaction conditions: 37° C. for 90 minutes

| | | | | | | | Acidic | | |
|---|---|---|---|---|---|---|---|---|---|
| | | | | Neutral | Acidic | Acidic/ | protease | | |
| | | | | protease | protease | neutral | use activity | Total | BCAA |
| | | | Digestive | use | use | protease | per 1 g of | FAA | content |
| | | | enzyme agent | activity | activity | activity | protein | (*1) | (*2) |
| | Origin | | name | [U] | [U] | ratio | [U/g] | [mg/L] | [%] |
| Comparative | — | | — | — | — | — | — | 640 | 10.7 |
| Example 1 | | | | | | | | | |
| Example 1 | Koji | Aspergillus | ASPSDU-pine | 3000 | 6800 | 2.26 | 2500 | 1810 | 32.4 |
| | mold | oryzae | | | | | | | |
| Example 2 | | Aspergillus | Protease M | 3000 | 2200 | 0.72 | 800 | 1770 | 31.9 |
| | | oryzae | Amano SD | | | | | | |
| Example 3 | | Aspergillus | ProteAX | 3000 | 300 | 0.093 | 100 | 910 | 18.0 |
| | | oryzae | | | | | | | |
| Example 4 | | Aspergillus | Acidic protease UF | 3000 | 3100 | 1.02 | 1100 | 990 | 22.8 |
| | | niger | Amano SD | | | | | | |
| Example 5 | | Aspergillus | Protease P | 3000 | 100 | 0.027 | 40 | 700 | 14.0 |
| | | melleus | Amano 3SD | | | | | | |
| Example 6 | | Rhizopus | Peptidase R | 3000 | 4100 | 1.37 | 1500 | 1280 | 24.6 |
| | | oryzae | | | | | | | |
| Comparative | Bacte- | Bacillus | Protin SD-AY10 | 3000 | 30 | 0.01 | 10 | 610 | 11.7 |
| Example 2 | rium | licheniformis | | | | | | | |
| Comparative | | Bacillus | Protin SD-NY10 | 3000 | 10 | 0.003 | 0 | 620 | 10.1 |
| Example 3 | | amyloliquefaciens | | | | | | | |
| Comparative | | Geobacillus | Thermoase PC10F | 3000 | 0 | 0.001 | 0 | 650 | 10.3 |
| Example 4 | | stearothermophilus | | | | | | | |
| Comparative | Vege- | Pineapple | Bromelain F | 3000 | 260 | 0.09 | 100 | 620 | 10.8 |
| Example 5 | table | | | | | | | | |
| Comparative | | Papaya | Papain W-40 | 3000 | 280 | 0.09 | 100 | 610 | 10.7 |
| Example 6 | | | | | | | | | |
| Comparative | Animal | Pig | Pepsin | 3000 | 52000 | 17.33 | 19000 | 680 | 11.9 |
| Example 7 | | | | | | | | | |
| Comparative | | Pig | Trypsin | 3000 | 30 | 0.01 | 10 | 640 | 10.9 |
| Example 8 | | | | | | | | | |

(*1) Total amount (unit: mg/L) of free amino acids
(*2) Proportion (unit: %) of amount (unit: mg/L) of free BCAAs in total amount (unit: mg/L) of free amino acids

As apparently shown from Table 1, in the digestive enzyme agents comprising a protease derived from a source other than the koji mold (Comparative Examples 2 to 8), the BCAA liberation amount was equivalent to that in the case of not using the digestive enzyme agent (Comparative Example 1), whereas in the digestive enzyme agents comprising a koji mold-derived protease (Examples 1 to 6), the liberation of BCAAs was effectively promoted. That is, it could be found that the digestive enzyme agent comprising a koji mold-derived protease had an effect of promoting the liberation of BCAAs.

As a result of using each digestive enzyme agent so that the amount of the neutral protease was the same, it was recognized that the effect of promoting the liberation of BCAAs tends to be higher as the amount of the acidic protease increases, and thus it was found that the action of the koji mold-derived acidic protease particularly effectively acts in BCAA liberation promotion. Particularly, it was recognized that, in the digestive enzyme agents comprising an Aspergillus oryzae-derived protease (Examples 1 to 3), the digestive enzyme agent comprising an Aspergillus niger-derived protease (Example 4), and the digestive enzyme agent comprising a Rizopus oryzae-derived protease (Example 5), the proportion of free BCAAs is high. In the digestive enzyme agent in which the proportion of free BCAAs is higher as the amount of the acidic protease used in the digestive enzyme agent per 1 g of the substrate protein and which comprises an Aspergillus oryzae-derived protease or a Rizopus oryzae-derived protease, it was recognized that, in Examples 1, 2, and 6 in which the amount of the acidic protease used in the digestive enzyme agent per 1 g of the substrate protein is equal to or more than a predetermined amount, free BCAAs are obtained at a proportion exceeding the weight ratio of BCAAs (22.9%) contained in the beef thigh meat protein as a substrate and a particularly remarkable effect of promoting the liberation of BCAAs is exhibited.

### Test Example 2

### Test Substrate

As a test substrate, 13 g of finely ground (3 mm ground) beef thigh meat (lean tissue) that was the same as in Test Example 1 was prepared.

### Digestive Enzyme Agent

A digestive enzyme agent having the composition shown in Table 2 (Example 7) was used. The digestive enzyme agent of Example 7 comprises ASPSDU-pine (digestive enzyme agent comprising a large amount of an Aspergillus oryzae-derived acidic protease) used in Example 1 and Aspergillus oryzae-derived protease contained in Biodiastase 2000 (comprising an acidic protease and a neutral protease). The amount of the digestive enzyme agent of Example 7 used was set so that the protease activity would be an amount of 3000 U as measured by an enzyme activity measurement method (measurement pH: 6.0) based on the Folin method shown in Example 1. For the digestive enzyme agent of 3000 U of the protease, the activity as measured by an enzyme activity measurement method (measurement pH: 3.0) based on the Folin method shown in Example 1 was obtained as the acidic protease activity (unit: U). The ratio of the acidic protease activity (unit: U) to 3000 U of the protease measured at a pH of 6.0 was regarded as the acidic protease ratio. These measurement results are shown in Table 2.

### Digestion Experiment in Digestive System Simulating Stomach

A digestion experiment was performed in the same manner as in Example 1 using the test substrate and the digestive enzyme agent described above to determine the total amount (mg/L) of free amino adds and the proportion (%) of the amount (mg/L) of free BCAAs in the total amount (mg/L) of free amino acids. Results are shown in Table 2.

TABLE 2
Substrate: beef thigh meat 13 g (substrate protein: 2.73 g), reaction conditions: 37° C. for 90 minutes

| | | | | | | Acidic/ | Acidic | | |
|---|---|---|---|---|---|---|---|---|---|
| | | | | Neutral | Acidic | neutral | protease use | Total | BCAA |
| | | | | protease | protease | protease | activity per | FAA | content |
| | Digestive | | | use activity | use activity | activity | 1 g of | (*1) | (*2) |
| | enzyme agent name | | | [U] | [U] | ratio | protein [U/g] | [mg/L] | [%] |
| Comparative | — | | | — | — | — | — | 700 | 14.4 |
| Example 9 | | | | | | | | | |
| Example 7 | Biodiastase 2000 | 60 | mg/g | 3000 | 4740 | 1.58 | 1800 | 1950 | 31.7 |
| | ASPSDU-pine | 300 | mg/g | | | | | | |
| | Lipase AP6 | 62.5 | mg/g | | | | | | |
| | Cellulase AP3 | 60 | mg/g | | | | | | |

(*1) Total amount (unit: mg/L) of free amino acids
(*2) Proportion (unit: %) of amount (unit: mg/L) of free BCAAs in total amount (unit: mg/L) of free ammo acids

As apparently shown from Table 2, it was recognized that the digestive enzyme agent comprising a koji mold-derived protease (Example 7) can liberate many BCAAs even when other digestive enzymes are contained. It was recognized that, in the digestive enzyme agent of Example 7, free BCAAs are obtained at a proportion exceeding the weight ratio of BCAAs (22.9%) contained in the beef thigh meat protein as a substrate and a particularly remarkable effect of promoting the liberation of BCAAs is exhibited.

### Test Example 3

### Test Substrate

As a test substrate, 13 g of green soybean was used The protein weight ratio of general green soybean is about 9%, and the weight ratio of BCAAs to the total amino acid weight is about 17%.

### Digestive Enzyme Agent

Digestive enzyme agents having the compositions shown in Table 3 were used.

### Digestion Experiment in Digestive System Simulating Stomach

In a 100 mL Erlenmeyer flask, 13 g of green soybean as a test substrate and 100 mL of artificial gastric juice (50 mmol/L NaCl, 2 mmol/L KCl, 0.18 mmol/L CaCl₂, 13% McIlvaine buffer solution (pH 5.0)) were placed and allowed to stand at 37° C. for 30 minutes, a digestive enzyme agent corresponding to 3,000 U of protease activity (pH 6.0) was added thereto, a stirrer bar (3.5 cm) was put thereinto, and the mixture was stirred at 250 rpm for 90 minutes. From 5 minutes to 65 minutes after the start of the reaction, 0.54 mL of 1 mol/L hydrochloric acid was added every 10 minutes. After 90 minutes, the mixture was allowed to stand in the boiling water bath for 10 minutes. After standing in the boiling water bath, the reaction solution was subjected to mesh filtration (2 mm square), and the obtained filtrate was centrifuged at 10,000 rpm for 10 minutes, thereby obtaining a supernatant containing free amino acids. As a control (Comparative Example 10), the same operation was performed except that the digestive enzyme agent was not added, thereby obtaining a supernatant.

The obtained supernatant was diluted 25-fold with water and filtered through a filter (0.45 μm), and then the amount of free amino acids was analyzed with an amino acid analyzer (amino acid analysis using Agilent 1260 Infinity II LC system) according to the protocol. The total amount (mg/L) of free amino acids thus obtained and the proportion (%) of the amount (mg/L) of free BCAAs in the total amount (mg/L) of free amino acids are shown in Table 3.

TABLE 3
Substrate: green soybean 13 g, reaction conditions: 37° C. for 90 minutes

| | | | | Neutral | Acidic | Acidic/ | | |
|---|---|---|---|---|---|---|---|---|
| | | | | protease | protease | neutral | Total | BCAA |
| | | | Digestive | use | use | protease | FAA | content |
| | | | enzyme agent | activity | activity | activity | (*1) | (*2) |
| | Origin | | name | [U] | [U] | ratio | [mg/L] | [%] |
| Comparative | — | | — | — | — | — | 710 | 3.3 |
| Example 10 | | | | | | | | |
| Example 8 | Koji mold | Aspergillus oryzae | ASPSDU-pine | 3000 | 6800 | 2.3 | 1190 | 14.6 |
| Example 9 | | Aspergillus oryzae | Protease M Amano SD | 3000 | 2200 | 0.7 | 1090 | 14.4 |
| Example 10 | | Aspergillus oryzae | ProteAX | 3000 | 300 | 0.1 | 910 | 7.6 |
| Example 11 | | Aspergillus melleus | Protease P Amano 3SD | 3000 | 100 | 0.03 | 870 | 5.6 |
| Comparative | Bacterium | Bacillus licheniformis | Protin SD-AY10 | 3000 | 30 | 0.01 | 780 | 3.4 |
| Example 11 | | | | | | | | |
| Comparative | | Bacillus | Protin SD-NY10 | 3000 | 10 | 0.003 | 740 | 3.3 |
| Example 12 | | amyloliquefaciens | | | | | | |
| Comparative | | Geobacillus | Thermoase PC10F | 3000 | 0 | 0.001 | 710 | 3.4 |
| Example 13 | | stearothermophilus | | | | | | |
| Comparative | Vegetable | Pineapple | Bromelain F | 3000 | 260 | 0.1 | 720 | 3.5 |
| Example 14 | | | | | | | | |
| Comparative | | Papaya | Papain W-40 | 3000 | 280 | 0.1 | 760 | 3.3 |
| Example 15 | | | | | | | | |
| Comparative | Animal | Pig | Pepsin | 3000 | 52000 | 17.3 | 790 | 4.4 |
| Example 16 | | | | | | | | |
| Comparative | | Pig | Trypsin | 3000 | 30 | 0.01 | 690 | 3.4 |
| Example 17 | | | | | | | | |

(*1) Total amount (unit: mg/L) of free amino acids
(*2) Proportion (unit: %) of amount (unit: mg/L) of free BCAAs in total amount (unit: mg/L) of free amino acids

As apparently shown from Table 3, in the digestive enzyme agents comprising a protease derived from a source other than the koji mold (Comparative Examples 11 to 17), the BCAA liberation amount was equivalent to that in the case of not using the digestive enzyme agent (Comparative Example 10), whereas in the digestive enzyme agents comprising a koji mold-derived protease (Examples 8 to 11), the liberation of BCAAs was effectively promoted. That is, it could be found that the digestive enzyme agent comprising a koji mold-derived protease had an effect of promoting the liberation of BCAAs.

As a result of using each digestive enzyme agent so that the amount of the neutral protease was the same, it was recognized that the effect of promoting the liberation of BCAAs tends to be higher as the amount of the acidic protease increases, and thus it was found that the action of the koji mold-derived acidic protease particularly effectively acts in BCAA liberation promotion. It was recognized that, in Examples 8 and 9 in which the amount of the acidic protease is large, free BCAAs are obtained at a proportion close to the weight ratio of BCAAs (about 17%) generally contained in the green soybean protein as a substrate although BCAAs tend to be particularly hardly liberated from the green soybean as a vegetable protein food material as compared with the meat of livestock, and a particularly remarkable effect of promoting the liberation of BCAAs is exhibited.

### Test Example 4

### Test Substrate

As a test substrate, 5 g of Soya flour FT-N (soybean powder manufactured by The Nisshin OilliO Group, Ltd.) or 5 g of LYSAMINE GPS (pea protein powder manufactured by Roquette Frères) was prepared. The weight ratio of BCAAs to the total amino acid weight of general soybean is about 17%, and the weight ratio of BCAAs to the total amino acid weight of general pea is about 17%.

### Digestive Enzyme Agent

Digestive enzyme agents having the compositions shown in Table 4 and Table 5 were used.

### Digestion Experiment in Digestive System Simulating Stomach

In a 100 mL Erlenmeyer flask, 5 g of soybean powder or 5 g of pea protein powder as a test substrate and 100 mL of artificial gastric juice (50 mmol/L NaCl, 2 mmol/L KCl, 0.18 mmol/L CaCl₂, 13% McIlvaine buffer solution (pH 5.0)) were placed and allowed to stand at 37° C. for 30 minutes, a digestive enzyme agent corresponding to 1,500 U of protease activity (pH 6.0) was added thereto, a stirrer bar (3.5 cm) was put thereinto, and the mixture was stirred at 250 rpm for 90 minutes. From 5 minutes to 65 minutes after the start of the reaction, 0.54 mL of 1 mol/L hydrochloric acid was added every 10 minutes. After 90 minutes, the mixture was allowed to stand in the boiling water bath for 10 minutes. After standing in the boiling water bath, the reaction solution was subjected to mesh filtration (2 mm square), and the obtained filtrate was centrifuged at 10,000 rpm for 10 minutes, thereby obtaining a supernatant containing free amino acids. In the same manner, the same operation was performed except that 5 g of LYSAMINE GPS (pea protein powder manufactured by Roquette Frères) was used as a test substrate, thereby obtaining a supernatant. As a control (Comparative Examples 18 and 24), the same operation was performed except that the digestive enzyme agent was not added, thereby obtaining a supernatant.

The obtained supernatant was diluted 25-fold with water and filtered through a filter (0.45 μm), and then the amount of free amino acids was analyzed with an amino acid analyzer (amino acid analysis using Agilent 1260 Infinity II LC system) according to the protocol. The total amount (mg/L) of free amino acids thus obtained and the proportion (%) of the amount (mg/L) of free BCAAs in the total amount (mg/L) of free amino acids are shown in Table 4 and Table 5.

TABLE 4
Substrate: soybean powder 5 g, reaction conditions: 37° C. for 90 minutes

| | | | | Neutral | Acidic | Acidic/ | | |
|---|---|---|---|---|---|---|---|---|
| | | | | protease | protease | neutral | Total | BCAA |
| | | | Digestive | use | use | protease | FAA | content |
| | | | enzyme agent | activity | activity | activity | (*1) | (*2) |
| | Origin | | name | [U] | [U] | ratio | [mg/L] | [%] |
| Comparative | — | | — | — | — | — | 240 | 4.9 |
| Example 18 | | | | | | | | |
| Example 12 | Koji mold | Aspergillus oryzae | ASPSDU- pine | 1500 | 3400 | 2.3 | 1030 | 29.2 |
| Comparative | Bacterium | Bacillus | Protin SD- NY10 | 1500 | 5 | 0.003 | 240 | 5.8 |
| Example 19 | | amyloliquefaciens | | | | | | |
| Comparative | Vegetable | Pineapple | Bromelain F | 1500 | 130 | 0.1 | 260 | 6.0 |
| Example 20 | | | | | | | | |
| Comparative | | Papaya | Papain W-40 | 1500 | 140 | 0.1 | 260 | 7.4 |
| Example 21 | | | | | | | | |
| Comparative | Animal | Pig | Pepsin | 1500 | 26000 | 17.3 | 290 | 6.9 |
| Example 22 | | | | | | | | |
| Comparative | | Pig | Trypsin | 1500 | 15 | 0.01 | 220 | 5.8 |
| Example 23 | | | | | | | | |

(*1) Total amount (unit: mg/L) of free amino acids
(*2) Proportion (unit: %) of amount (unit: mg/L) of free BCAAs in total amount (unit: mg/L) of free amino acids

TABLE 5
Substrate: pea protein powder 5 g, reaction conditions: 37° C. for 90 minutes

| | | | | Neutral | Acidic | Acidic/ | | |
|---|---|---|---|---|---|---|---|---|
| | | | | protease | protease | neutral | Total | BCAA |
| | | | Digestive | use | use | protease | FAA | content |
| | | | enzyme agent | activity | activity | activity | (*1) | (*2) |
| | Origin | | name | [U] | [U] | ratio | [mg/L] | [%] |
| Comparative | — | | — | — | — | — | 700 | 4.8 |
| Example 24 | | | | | | | | |
| Example 13 | Koji mold | Aspergillus oryzae | ASPSDU-pine | 1500 | 3400 | 2.3 | 1065 | 17.9 |
| Comparative | Bacterium | Bacillus | Protin SD-NY10 | 1500 | 5 | 0.003 | 610 | 4.5 |
| Example 25 | | amyloliquefaciens | | | | | | |
| Comparative | Vegetable | Papaya | Papain W-40 | 1500 | 140 | 0.1 | 770 | 4.9 |
| Example 26 | | | | | | | | |
| Comparative | Animal | Pig | Pepsin | 1500 | 26000 | 17.3 | 780 | 6.5 |
| Example 27 | | | | | | | | |

(*1) Total amount (unit: mg/L) of free amino acids
(*2) Proportion (unit: %) of amount (unit: mg/L) of free BCAAs in total amount (unit: mg/L) of free amino acids

As apparently shown from Table 4 and Table 5, in the digestive enzyme agents comprising a protease derived from a source other than the koji mold (Comparative Examples 19 to 23 and Comparative Examples 25 to 27), the BCAA liberation amount was equivalent to that in the case of not using the digestive enzyme agent (Comparative Example 18 and Comparative Example 24), whereas in the digestive enzyme agents comprising a koji mold-derived protease (Example 12 and Example 13), a remarkable BCAA liberation effect was recognized to the same extent as or more than the weight ratio of BCAAs (about 17%) generally contained in the green soybean protein or pea as the substrate. That is, it could be found that the digestive enzyme agent comprising a koji mold-derived protease had an effect of promoting the liberation of BCAAs.

1. A digestive enzyme agent comprising a protease derived from a koji mold.

2. The digestive enzyme agent according to claim 1, wherein the koji mold is Aspergillus oryzae and/or Aspergillus niger.

3. The digestive enzyme agent according to claim 1, wherein the protease comprises an acidic protease.

4. The digestive enzyme agent according to claim 3, wherein the acidic protease is used in an amount of 10 U or more per 1 g of a substrate protein.

5. The digestive enzyme agent according to claim 1, wherein the digestive enzyme agent is used for digestion of meat.

6. The digestive enzyme agent according to claim 1, wherein the digestive enzyme agent is used for digestion of a vegetable protein.

7. A drug for oral administration for promoting liberation of a protein into branched-chain amino acids, comprising the digestive enzyme agent according to claim 1.

8. A food additive for promoting liberation of a protein into branched-chain amino acids, comprising the digestive enzyme agent according to claim 1.

9. A food or drink for promoting liberation of a protein into branched-chain amino acids, comprising the digestive enzyme agent according to claim 1.
